(19) United States
(12) Patent Application Publication
(10) Pub. No.: US 2015/0218599 A1
(43) Pub. Date: Aug. 6, 2015

(54) METHODS FOR PRODUCTION OF ALCOHOLS FROM CARBOXYLIC ACIDS VIA FERMENTATION

(71) Applicant: Cornell University, Ithaca, NY (US)

(72) Inventors: Largus T. Angenent, Ithaca, NY (US); Hanno Richter, Ithaca, NY (US)

(21) Appl. No.: 14/419,080

(22) PCT Filed: Aug. 8, 2013
(86) PCT No.: PCT/US2013/054125

§ 371 Date: Feb. 2, 2015

Related U.S. Application Data
(60) Provisional application No. 61/680,942, filed on Aug. 8, 2012.

(51) Int. Cl. C12P 7/22; C12P 7/18; C12P 7/04; C12P 7/16

(52) U.S. Cl. CPC C12P 7/22; C12P 7/16; C12P 7/18; C12P 7/04

(57) ABSTRACT

Methods for obtaining a product comprising a substituted or unsubstituted C₃ to C₁₀ alcohol from a substituted or unsubstituted C₃ to C₁₀ carboxylic acid. The method comprises contacting a substituted or unsubstituted C₃ to C₁₀ carboxylic acid with a gaseous composition and a Chlostridia species to produce a substituted or unsubstituted C₃ to C₁₀ alcohol under an anaerobic environment.

### CROSS-REFERENCE TO RELATED APPLICATIONS

This application claims priority to U.S. Provisional Application No. 61/680,942, filed on Aug. 8, 2012, the disclosure of which is incorporated herein by reference.

### STATEMENT REGARDING FEDERALLY SPONSORED RESEARCH

This invention was made with government support under Grant Number DTOS59-07-G-00052 awarded by US Department of Transportation. The government has certain rights in the invention.

### FIELD OF THE DISCLOSURE

The present disclosure generally relates to the field of obtaining selected alcohols. More particularly, the present disclosure relates to methods for obtaining medium chain alcohols from carboxylic acids.

### BACKGROUND OF THE DISCLOSURE

Short-chain carboxylic acids generated by various mixed- or pure-culture fermentation processes can be valuable precursors for production of alcohols. Conversion of carboxylic acids into alcohols can be performed via catalytic hydrogenation or with strong chemical reducing agents. However, this reduction reaction costs electrons and energy and has been performed with anaerobic fermentation by adding sugar as a source.

### BRIEF SUMMARY OF THE DISCLOSURE

The present disclosure provides methods for producing alcohols from carboxylic acids, a gaseous composition comprising either i) carbon monoxide, ii) carbon monoxide and hydrogen, or iii) carbon dioxide and hydrogen, and a Clostridia species.

In an aspect, the method for obtaining a product comprising a substituted or unsubstituted C₃ to C₁₀ alcohol comprises the steps of: contacting a mixture of Clostridia species and substituted or unsubstituted C₃ to C₁₀ carboxylic acid with a gaseous composition comprising: i) carbon monoxide, ii) carbon monoxide and hydrogen, or iii) carbon dioxide and hydrogen; c) maintaining the reaction conditions such that the substituted or unsubstituted C₃ to C₁₀ alcohol is formed in the mixture, and at least 40% of the carbon from the exogenous substituted or unsubstituted C₃ to C₁₀ carboxylic acid is recovered as alcohol in the prescence of the Clostridia. It was observed that no meidiator or metal catalyst was needed for the production of the alcohols. In an embodiment, the steps of the method are carried out in the absence of a compound selected from the group consisting of viologen dyes, anthraquinone and other quinone dyes, triphenylmethane dyes, phthalocyanines, methane dyes, pyrrole dyes, pteridines and pteridones, flavines, or metal complexes of metals of secondary groups VI, VII and VIII. The production of alcohols according to the present method can be carried out in a batch setup or continuous setup

### DESCRIPTION OF THE DRAWINGS

FIG. 1 shows an example of an experimental setup for conversion of carboxylic acids into alcohols, using carboxydotrophic, ethanol-producing Clostridia with syngas. A) Setup with cultures continuously sparged with syngas for testing the ability to convert different carboxylic acids into alcohols. B) Cultures with a finite amount of syngas for determining the fermentation stoichiometry and cell yields during syngas fermentation without and with n-butyric acid.

FIG. 2 shows representative carboxylic acid reduction experiments with Clostridium ljungdahlii ERI-2 in medium with 15 mM carboxylic acid of different carbon chain length. Values were obtained from triplicate batch cultures with a constant supply of syngas. A) no acids, B) 15 mM propionic acid, C) 15 mM n-butyric acid, D) 15 mM n-valeric acid, E) 15 mM n-caproic acid, F) 15 mM isobutyric acid. OD_{(600 nm)} (X), pH (+), concentrations of acetic acid (□), propionic acid (▴, dashed line), n-butyric acid (, dashed line), n-valeric acid (♦, dashed line), n-caproic acid (⋄, dashed line), isobutyric acid (Δ, dashed line), ethanol (▪), propanol (▴), n-butanol (), n-pentanol (♦), n-hexanol (⋄), isobutanol (2-methy-1-propanol) (Δ). Error bars indicate standard deviation.

FIG. 3 shows representative batch fermentation with C. ljungdahlii ERI-2 in 1 L reactors without and with 15 mM n-butyric acid and a finite amount of syngas. A) OD₆₀₀ and pH without n-butyric acid, B) OD and pH with n-butyric acid, C) gas quantities without n-butyric acid, D) gas quantities with n-butyric acid, E) acetic acid and ethanol without n-butyric acid, F) acetic acid, ethanol, n-butyric acid, and n-butanol with n-butyric acid. OD₆₀₀ₙₘ (▪), pH (♦), total amount of acetic acid (▴), n-butyric acid (Δ), ethanol (), n-butanol (◯), quantities of CO (x), H₂ (⋄), and CO₂ (□). Error bars indicate one standard deviation.

FIG. 4 shows an example of a calculation of the ATP/CO yield of the Wood Ljungdahl pathway using two different approaches. Values are normalized to one mol of CO consumed. A) metabolic model for flux of carbon and electrons, and protons translocated via the Rnf complex of batch cultures without and with 15 mM n-butyric acid and a finite amount of syngas. Assumed is a proton/electron ratio of 1.0 at the Rnf complex (see text). B) energy required for alcohol and biomass formation is estimated based on 1 mol ATP required for conversion of 1 mol carboxylic acid to alcohol, and 6 mol ATP required per 10 grams dry weight biomass synthesized from carbon monoxide. All data were obtained during the growth phase (days 25-115 h).

FIG. 5 shows an example of a setup of two-stage continuous fermentation with cell and gas recycle. Solid lines: flow of liquid media; dotted lines: flow of substrate and exhaust gases. Abbreviations: 1-7 pumps; Ag agitation; BP bypass; E effluent reservoir; Ex exhaust; FT foam trap; G1, G2 gas recycle loops; HF hollow fiber module for cell recycle; M media reservoir; Per permeate; Ret retentate.

### DETAILED DESCRIPTION OF THE DISCLOSURE

The present disclosure provides methods for producing and, optionally, sequestering liquid alcohols from carboxylic acids, a gaseous composition comprising either i) carbon monoxide, ii) carbon monoxide and hydrogen, or iii) carbon dioxide and hydrogen and a Clostridia species.

Without intending to be bound by any particular theory it is considered that the reaction involved in an embodiment of the present disclosure is as follows:

6.7CO+2.0H₂→1.0 ethanol+0.1 acetic acid+0.1 n-butyric acid+4.7 CO₂+0.1 n-butanol+0.4 cell carbon (Eqn 1)

Compare this to the background reaction of syngas fermentation:

7.1CO+1.0H₂→1.0 ethanol+0.1 acetic acid+5.0 CO₂+0.4 cell carbon (Eqn 2)

In an aspect, the present disclosure provides methods for obtaining a product comprising a substituted or unsubstituted C₃ to C₁₀ alcohol. In one embodiment, the method comprises contacting a gaseous mixture comprising i) carbon monoxide, ii) carbon monoxide and hydrogen, or iii) carbon dioxide and hydrogen with a substituted or unsubstituted C₃ to C₁₀ carboxylic acid, and Clostridia species. In one embodiment, the method for obtaining a product comprising a substituted or unsubstituted C₃ to C₁₀ alcohol comprises the steps of: a) providing in a vessel a mixture comprising i) a substituted or unsubstituted C₃ to C₁₀ carboxylic acid, and ii) Clostridia species; b) contacting the mixture of step a) with a gaseous composition comprising: i) carbon monoxide, ii) carbon monoxide and hydrogen, or iii) carbon dioxide and hydrogen; c) maintaining the reaction conditions such that the substituted or unsubstituted C₃ to C₁₀ alcohol is formed in the mixture. In an embodiment, at least 40% of the carbon from the exogenous carboxylic acid is recovered as alcohol in the presence of the Clostridia. In various embodiments, the disclosure provides a method for obtaining a product comprising a substituted or unsubstituted C₃ to C₁₀ alcohol where at least 40%, 50%, 60%, 70%, 80%, or 90% of the carbon from the exogenous carboxylic acid is recovered as the corresponding alcohol formed in the presence of the Clostridia. In various other embodiments, the disclosure provides a method for obtaining a product comprising a substituted or unsubstituted C₃ to C₁₀ alcohol where at least 90%, 91%, 92%, 93%, 94%, 95%, 96%, 97%, 98%, 99%, 100% of the carbon from the exogenous carboxylic acid is recovered as the corresponding alcohol formed in the presence of the Clostridia.

The method of the present disclosure is conducted in a vessel under anaerobic conditions. For example, anaerobic conditions can be achieved by sealing the vessel and the system except to allow products (both liquid products and gas products) to be separated or escape. As used herein “vessel” refers to a reaction flask, reactor, or any other container and is meant to refer to a single vessel or more than one vessel (e.g., reactor network) for carrying out different stages of the reaction or all stages of the reaction. The reactants or contents of the vessel can be made of a number of different materials. For example, the vessel can be glass or stainless steel and constructed as to prevent diffusion through fittings and withstand pressurization. The vessel can be mixed periodically to promote carboxylic acid-microorganism contact.

The term “carboxylic acid” as used herein, unless otherwise stated, is meant to refer to linear and branched carboxylic acids or the salts of the corresponding carboxylic acids. The carboxylic acids can be derived from a variety of sources. The carboxylic acids can have from 3 carbons to 10 carbons and the linear or branched carbon chain can be substituted with groups such as aryl groups (e.g., phenyl group), alkoxy groups (e.g., methoxy), or hydroxy groups. The carboxylic acids can have unsaturation (e.g., conjugated (α,β-unsaturated carboxylic acid) or unconjugated (alkene)). The carboxylic acids can have more than one carboxylic acid functionality (i.e., a dicarboxylic acid). The carboxylic acids can be obtained from commercial sources, synthesized by methods known in the art or obtained from another fermentation process. For example, the carboxylic acid used can be propionic acid, n-butyric acid, n-valeric acid, n-caproic acid, enanthic acid, caprylic acid, pelargonic acid, capric acid, isobutyric acid, benzoic acid, phenylacetic acid, phenylpropionic acid, phenylbutyric acid, 3-methoxy-4-hydroxybenzoic acid, 3-methoxy-4-hydroxyphenylacetic acid, cinnamic acid, lactic acid, 2-methylbutyrate, 2-methyl-2-buteneoate, glutaric acid, succinic acid, adipic acid, or combinations thereof. Thus, the substituted C₃ to C₁₀ alcohols formed from these substituted C₃ to C₁₀ carboxylic acids will have the same substitution pattern (may be referred to herein as a corresponding alcohol). For example, the substituted or unsubstituted C₃ to C₁₀ alcohols formed by the method of the present disclosure are n-propanol, n-butanol, n-pentanol, n-hexanol, n-heptanol, n-octanol, n-nonanol, n-decanol, isobutanol, benzyl alcohol, 4-phenylbutan-1-ol, (E)-3-phenylprop-2-en-1-ol, 4-(hydroxymethyl)-2-methoxyphenol, 2-phenylethanol, 4-(2-hydroxyethyl)-2-methoxyphenol, 3-phenylpropan-1-ol, propane-1,2-diol, (E)-2-methylbut-2-en-1-ol, 2-methylbutan-1-ol, pentane-1,5-diol, butane-1,4-diol, hexane-1,6-diol, or combinations thereof.

The gaseous composition (source of carbon and source of electrons) of the method comprises one of the following: i) carbon monoxide, ii) carbon monoxide and hydrogen, or iii) carbon dioxide and hydrogen. When carbon monoxide and hydrogen are used, the gaseous mixture is referred to as “syngas.” In addition to these two gaseous components, syngas can optionally contain inert components that are derived from the gasification of coal, oil residues, waste or biomass. Methods for removing these other inert components from syngas are known in the art. In one embodiment, the syngas used in the present disclosure comprises hydrogen gas and carbon monoxide gas. The gases can be added separately or combined and added to the mixture. The gasses used can be of varying purity. In an embodiment, the ratio of CO:H₂ can be from 1:0 to 1:2 including all ratios therebetween. In an embodiment, the composition of the syngas used is 60% carbon monoxide and 35% hydrogen. The gaseous composition used in the methods can be used in a constant flow process or can exist in a static environment (i.e., finite amount) in the vessel. The flow rate of the gaseous composition will depend on the size of the vessel used. In various embodiments, the gaseous composition flow rate is from 10 mL min⁻¹ L⁻¹_{reactor volume} to 100 mL min⁻¹ L⁻¹_{reactor volume} including all values to the 0.10 mL min⁻¹ L⁻¹_{reactor volume} and all ranges therebetween. In another embodiment, the gaseous composition flow rate is from 1 mL min⁻¹ L⁻¹_{reactor volume} to 10 mL min⁻¹ L⁻¹_{reactor volume} including all values to the 0.10 mL min⁻¹ L⁻¹_{reactor volume} and all ranges therebetween. In one embodiment, the only source of hydrogen gas is from syngas. In one embodiment, the source of hydrogen gas is not from a sugar (e.g., glucose). Various pressures of the gaseous composition can be used within the vessel. In an embodiment, the gas pressure is ambient pressure. In various other embodiments, the pressure within the vessel of the gaseous composition is from 1 atm to 10 atm including all values to the 0.01 atm and ranges therebetween.

In an embodiment, no mediator (i.e., an organic reducing agent) is used. In an embodiment, the reaction mixture does not contain or is not exposed to a mediator. In various embodiments, the mediator is less than 0.5 mM, 0.1 mM, 0.05 mM, or 0.01 mM. In an embodiment, the reaction mixture does not contain or is not exposed to 0.5 mM, 0.1 mM, 0.05 mM, 0.01 mM, or any amount of mediator selected from the group consisting of viologen dyes, anthraquinone and other quinone dyes, triphenylmethane dyes, phthalocyanines, methane dyes, pyrrole dyes, pteridines and pteridones, flavines, or metal complexes of metals of secondary groups VI, VII and VIII.

In an embodiment, cells are in the growth phase. In an embodiment, more than 50%, 60%, 70%, 80%, 90%, 95%, 99% of the bacterial cells are in the growth phase.

In an embodiment, the method further comprises the step of separating at least a portion of the substituted or unsubstituted C₃ to C₁₀ alcohol from the mixture. Examples of suitable methods of separation of the product alcohols from the mixture comprise ordinary distillation, azeotropic distillation, reflux distillation, gas stripping, pervaporation, extractive distillation (i.e., with liquid solvent, with a dissolved salt, with a mixture of liquid solvent and dissolved salt, with an ionic liquid, or with hyperbranched polymers), liquid-liquid extraction, adsorption (i.e., vapor-phase, liquid-phase), and membrane separation methods (i.e., hydrophilic membrane, hydrophobic membrane, or vacuum membrane distillation-bioreactor hybrid). Other alcohol separation technologies are known in the art.

The reaction conditions of the method can vary. In an embodiment, the temperature of the reaction within the vessel is from 20° C. to 45° C. including all values to the ° C. and ranges therebetween. In another embodiment, the temperature is from 30° C. to 40° C. or from 35° C. to 37° C. In an embodiment, the pH of the reaction is maintained from 4.0 to 6.5, including all values to the tenth decimal place and ranges therebetween. Thus, in an embodiment, this reaction is conducted at 20° C. to 45° C. and a pH of from 4.0 to 6.5. Thus, in an embodiment, this reaction is conducted at 30° C. to 40° C. and a pH of from 4.0 to 6.5. Thus, in an embodiment, this reaction is conducted at 35° C. to 37° C. and a pH of from 4.0 to 6.5. Without intending to be bound by any particular theory, it is realized that maintaining this pH range minimizes cell death and sporulation. Also, it is realized that maintaining these temperature conditions controls the ethanol concentration (e.g., keeps it from becoming too high or too low) and also allows for increased enzymatic activity and increased rates of product alcohol production. The pH of the reaction mixture can be controlled by addition of carboxylic acid substrate (C₃ to C₁₀ unsubstituted or substituted carboxylic acid), buffer solution, a pH auxostat, or by addition of acid/base to the mixture to reach the desired pH of the mixture. In an embodiment, the reaction is run for at least 24 hours. In another embodiment, the specific rates of formation of product alcohols are from 0.5 to 1.0 mmol per gram cell dry weight per minute including all values to the 0.01 mmol per gram cell dry weight per minute and all ranges therebetween.

The method can also be conducted as a two-stage process. In the 1^{st} stage of the reaction, the pH is maintained at from 5.0 to 6.5, including all values to the tenth decimal place and ranges therebetween to promote growth of the Clostridia species. In another stage of the reaction, the pH is maintained at from 4.0 to 5.5, or from 4.5 to 5.5 to promote the formation of the product alcohols. In an embodiment, in a 2-stage system the reaction can be run for 14 days to 90 days including all days and ranges therebetween. In another embodiment, the 2-stage system can be carried out for 90 days to a year including all days and ranges therebetween. In an embodiment, the 2-stage system can be carried out indefinitely (e.g., years).

The term “Wood-Ljungdahl pathway” (also the reductive acetyl-CoA pathway) is a term used to define a set of biochemical reactions used by some bacteria and archaea. The Wood-Ljungdahl pathway enables certain organisms to use H₂ as an electron donor and CO₂ as an electron acceptor as well as a building block for biosynthesis. In the Wood-Ljungdahl pathway, CO₂ is reduced to CO, which is then converted to acetyl coenzyme A. Two key enzymes participate, CO Dehydrogenase and acetyl-CoA synthase. The former catalyzes the reduction of the CO₂ and the latter combines the resulting CO with a methyl group to give acetyl CoA.

In the method of the present disclosure, several different anaerobic carboxydotrophic microorganisms can be used. For example, the bacteria can be mesophillic bacteria, mesophillic archaea, thermophillic bacteria, thermophillic archaea, or a combination thereof. In various embodiments, the anaerobic carboxydotrophic microorganism is selected from the group consisting of Clostridium ljungdahlii (e.g., ERI-2, PETC, C-01), Clostridium ragsdalei (e.g., P11), Clostridium autoethanogenum, Clostridium carboxidivorans, Clostridium coskatii, Oxobacter pfennigii, Peptostreptococcus productus, Acetobacterium woodii, Eubacterium limosum, Butyribacterium methylotrophicum, Rubrivivax gelatinosus, Rhodopseudomonas palustris P4, Rhodospirillum rubrum, Citrobacter sp Y19, Methanosarcina barkeri, Methanosarcina acetivorans strain C2A, Moorella thermoacetica, Moorella thermoautotrophica, Moorella strain AMP, Carboxydothermus hydrogenoformans, Carboxydibrachium pacificus, Carboxydocella sporoproducens, Carboxydocella thermoautotrophica, Thermincola carboxydiphila, Thermincola ferriacetica, Thermolithobacter carboxydivorans, Thermosinus carboxydivorans, Desulfotomaculum kuznetsovii, Desulfotomaculum thermobenzoicum subsp., thermosyntrophicum, Desulfotomaculum carboxydivorans, Methanothermobacter thermoautotrophicus, Thermococcus strain AM4, Archaeoglobus fulgidus, Alkalibaculum bacchi CP11, CP13, and CP15, or a combination thereof. In an embodiment, a carboxydotrophic microorganism in which the Wood-Ljungdahl pathway is non-functional is used. This can be accomplished by genetic modification of the microorganism. For example, the gene or genes encoding one or more of the enzymes in the Wood-Ljungdahl pathway hydrogen (i.e., formate dehydrogenase, formyl-THF synthetase, methyl-THF cyclohydrogenase, methylene-THF reductase, methyltransferase, Acetyl-CoA synthase (ACS)), other than the initial dehydrogenases that oxidize CO or for the production of Acetyl-CoA are non-functional or may be inactivated or deleted to block or shut down the Wood-Ljungdahl pathway. In one embodiment, a Clostridia species can be used. For example, the Clostridia species can be a wild type Clostridia species. In an embodiment, the microorganisms are Clostridium ljungdahlii ERI-2, Clostridium ljungdahlii PETC, Clostridium ljungdahlii C-01, Clostridium ragsdalei P11, or a combination thereof. In an embodiment, the microorganisms are not Clostridium thermoaceticium (DSM 521), Clostridium aceticium (DSM 1496), Clostrium formicoacetium (DSM 92), Butyribacterium methylotrophicum (DSM 3468), Acetobacterium woodii (DSM 1030), Desulfobacterium autotrophicum (DSM 3382), or Desulfobacterium limosum (20402). In an embodiment, there are no non-biological metal catalysts capable of converting the exogenous carboxylic acids to their corresponding alcohols in the mixture. In another embodiment, there are no inorganic metal catalysts capable of converting the exogenous carboxylic acids to their corresponding alcohols in the mixture.

The culture medium (inoculum source) can contain aqueous stock solutions for minerals, vitamins, and trace metals. The aqueous mineral stock solution can contain sodium chloride, ammonium chloride, potassium chloride, potassium phosphate monobasic, magnesium sulfate, and calcium chloride. The aqueous vitamin stock solution can contain, pyridoxine, thiamine, riboflavin, calcium pantothenate, thioctic acid, amino benzoic acid, nicotinic acid, vitamin B12, biotin, folic acid, and MESNA (2-(N-morpholino)ethanesulfonic acid (sodium salt)). The aqueous trace metals stock solution can contain nitrilo triacetic acid, manganese sulfate, ferrous ammonium sulfate, cobalt chloride, zinc sulfate, copper chloride, nickel chloride, sodium molybdate, sodium selenite, and sodium tungstate. The final pH of the medium can be adjusted accordingly and yeast extract can be added. One having skill in the art would recognize the various ways to prepare a culture medium. Examples of culture medium preparation are provided in the examples that follow.

The method of the disclosure can be carried out continuously or semi-continuously (e.g., batch). Semi-continuously refers to batch cultures in terms of growth media that are continuously fed with the gaseous composition described herein. In one embodiment, the vessel is fed continuously with a growth medium comprising the Clostridia species and the gaseous composition described herein. The fermentation can be separated into two vessels (stages) that are connected in series regarding the flow of growth medium. Without intending to be bound by any particular theory, it is realized that the purpose of the first stage is to grow bacteria (biocatalyst), the purpose of the second stage is to use the grown biocatalyst to convert carboxylic acids into alcohols. In an embodiment, the dilution rate in the first vessel is lower than the specific maximum growth rate of the respective bacterium to avoid washout of cells and to accumulate biocatalyst in stage one and the dilution rate in the second vessel is adjusted in a manner that results in limitation of nutrients for cell growth in stage 2. In one embodiment, the dilution rate in the second vessel is 4× lower than in the first vessel. In an embodiment, the ratio of the dilution rate of stage 1:stage 2 is from 0.1 to 10 including all values to the 0.01 and ranges therebetween. In an embodiment, the method can be carried out indefinitely (e.g., years) or from 14 to 90 days including all days and ranges therebetween. In another embodiment, the method can be carried out from 90 days to a year including all days and ranges therebetween. Nonlimiting methods for carrying out a batch or continuous process are described in the examples that follow.

The products formed from the present method comprise a liquid component and a gaseous component. The liquid component can also contain, for example, acetic acid and ethanol, which are produced by the fermentation of the gaseous components of the reaction mixture. In an embodiment, less than 140 mM, 120 mM, 100 mM, 60 mM, 30 mM, 15 mM, 10 mM, or 5 mM of acetic acid is formed in the reaction mixture. In an embodiment, less than 170 mM, 150 mM, 100 mM, 60 mM, 30 mM, 15 mM, 10 mM, or 5 mM of ethanol is formed in the reaction mixture. In an embodiment, ethanol, acetic acid, or a combination thereof are not produced in detectable amounts.

In an embodiment, the method further comprises recycling and/or replenishing the gaseous component. In an embodiment, the method further comprises separating the gaseous component from the reaction mixture.

The gaseous component of the product can comprise hydrogen gas, carbon monoxide gas, carbon dioxide gas, or a combination thereof.

In an embodiment, the method is carried out continuously by feeding the vessel continuously or semi-continuously (e.g., batch) with carboxylic acid, a gaseous composition as described herein, and nutrients (e.g., vitamins and minerals) to feed the microorganisms.

In various examples, the method can convert n-butyric acid into n-butanol; n-valeric acid into n-pentanol, n-caproic acid into hexanol; isobutyric acid into isobutanol using syngas as the electron donor and energy source.

The steps of the method described in the various embodiments and examples disclosed herein are sufficient to produce the products of the present disclosure. Thus, in one embodiment, the method consists essentially of a combination of the steps of the method disclosed herein. In another embodiment, the method consists of such steps.

In an embodiment, the reaction mixture consists essentially of: a substituted or unsubstituted C₃ to C₁₀ carboxylic acid, Clostridia species and a gaseous composition of either: i) carbon monoxide, ii) carbon monoxide and hydrogen, or iii) carbon dioxide and hydrogen.

Therefore, in an aspect, the disclosure provides product made by the process disclosed herein. In one embodiment, the disclosure provides a liquid component comprising a substituted or unsubstituted alcohol having from 3 to 10 carbons. In various other embodiments, the method provides a liquid component comprising a substituted or unsubstituted alcohol having 3 carbons, 4 carbons, 5 carbons, 6 carbons, 7 carbons, 8 carbons, 9 carbons, 10 carbons, or a combination thereof.

In one embodiment, the disclosure provides a product comprising from 50% to 99% substituted or unsubstituted C₃, C₄, C₅, C₆, C₇, C₈, C₉, C₁₀ alcohol, including all values to the 0.1% and ranges therebetween. In one embodiment, the disclosure provides a product comprising greater than 99% or 99% to 100% substituted or unsubstituted C₃, C₄, C₅, C₆, C₇, C₈, C₉, C₁₀ alcohol.

The products (e.g., substituted or unsubstituted C₃ to C₁₀ alcohols) can be removed in-line in a continuous manner from the vessel. In one embodiment, at least a portion of the substituted or unsubstituted C₃, C₄, C₅, C₆, C₇, C₈, C₉, C₁₀ alcohols, or a combination thereof are removed from the mixture. In various other embodiments, at least 80%, 85%, 90%, 91%, 92%, 93%, 94%, 95%, 96%, 97%, 98%, 99% of the substituted or unsubstituted C₃, C₄, C₅, C₆, C₇, C₈, C₉, C₁₀ alcohols, or a combination thereof are removed from the mixture.

The substituted or unsubstituted C₃ to C₁₀ alcohols produced by the present method can be used for various applications (e.g., biofuels). For example, the alcohol produced by the method disclosed herein is n-propanol, n-butanol, n-pentanol, n-hexanol, n-helptanol, n-octanol, n-nonanol, n-decanol, isobutanol, benzyl alcohol, 4-phenylbutan-1-ol, (E)-3-phenylprop-2-en-1-ol, 4-(hydroxymethyl)-2-methoxyphenol, 2-phenylethanol, 4-(2-hydroxyethyl)-2-methoxyphenol, 3-phenylpropan-1-ol, propane-1,2-diol, (E)-2-methylbut-2-en-1-ol, 2-methylbutan-1-ol, pentane-1,5-diol, butane-1,4-diol, hexane-1,6-diol, or a combination thereof. Additionally, the alcohols can be converted into alkanes for biofuel. For example, the alcohols can be converted to alkanes by a subsequent process. Methods for converting alcohols to alkanes are known in the art.

In an aspect the disclosure provides a kit for obtaining a product comprising a substituted or unsubstituted C₃ to C₁₀ alcohol.

In an embodiment, the kit for obtaining a product comprising a substituted or unsubstituted C₃ to C₁₀ alcohol comprises: 1) a Clostridia species; 2) substituted or unsubstituted C₃ to C₁₀ carboxylic acid; 3) culture medium for the Clostridia or a composition for preparing said culture medium; and 4) instructions comprising one or more of the following: a) instructions for culturing the Clostridia; b) instructions for mixing of 1), 2) and 3); c) instructions for the introduction of a gaseous composition comprising: i) carbon monoxide, ii) carbon monoxide and hydrogen, or iii) carbon dioxide and hydrogen including the composition and the flow rate; and optionally, d) instructions for separation of substituted or unsubstituted C₃ to C₁₀ alcohol from the mixture.

In an embodiment, the kit contains a wild type Clostridia species. In various embodiments, the kit contains a Clostridia selected from the group consisting of Clostridium ljungdahlii ERI-2, Clostridium ljungdahlii PETC, Clostridium ljungdahlii C-01, Clostridium ragsdalei P11, Clostridium autoethanogenum, Clostridium carboxidivorans, Clostridium coskatii, or a combination thereof.

The following examples are presented to illustrate the present disclosure. They are not intended to limiting in any manner.

### Example 1

In this example, a biological conversion route for conversion of carboxylic acids into alcohols was explored. The potential of carboxydotrophic bacteria, such as Clostridium ljungdahlii and Clostridium ragsdalei, as biocatalysts for conversion of short-chain carboxylic acids into alcohols, using syngas as a source of electrons and energy is demonstrated. Acetic acid, propionic acid, n-butyric acid, isobutyric acid, n-valeric acid, and n-caproic acid were converted into their corresponding alcohols. Furthermore, biomass yields and fermentation stoichiometry from the experimental data were modeled to determine how much metabolic energy C. ljungdahlii generated during syngas fermentation. An ATP yield of 0.4-0.5 mol of ATP per mol CO consumed was calculated in the presence of hydrogen. The ratio of protons pumped across the cell membrane vs. electrons transferred from ferredoxin to NAD⁺ via the Rnf complex is suggested to be 1.0. Based on these results, provided are suggestions of how n-butyric acid to n-butanol conversion via syngas fermentation can be further improved.

All chemicals were purchased from Sigma-Aldrich, St. Louis, Mo., unless stated otherwise. Bacterial strain and growth conditions. Most experiments were conducted with C. ljungdahlii ERI-2 (ATTC 55380), which has been used successfully for syngas to ethanol fermentation. Some experiments were repeated with C. ragsdalei P11 (ATTC BAA-622). Bacteria were grown anaerobically at 35° C. in medium designed for efficient syngas fermentation. This medium is mostly based on ATCC medium 1754 but contains higher concentrations of the macronutrient elements. The yeast extract concentration was 0.5 g/L or 0.0 g/L as indicated for each experiment; the concentration of MES (2-(N-morpholino)ethanesulfonic acid) was 150 mM; carboxylic acids (propionic, n-butyric, isobutyric, n-valeric or n-caproic acid) were added to final concentrations of 15 mM before adjustment of the pH to 5.5. The syngas atmosphere was a synthetic blend of 60% [vol/vol]carbon monoxide, 35% hydrogen, and 5% carbon dioxide (Airgas East, Ithaca, N.Y.). Precultures of C. ljungdahlii or C. ragsdalei were grown in 250 ml serum bottles containing 20 ml medium with 0.5 g/L or 0.0 g/L yeast extract (the latter for inoculation of experiments with no yeast extract) and syngas in the headspace at a pressure of 28 psi with finite amounts of syngas. Precultures were maintained in an active state by weekly transfer of 2% into a fresh serum bottle with a headspace of finite syngas.

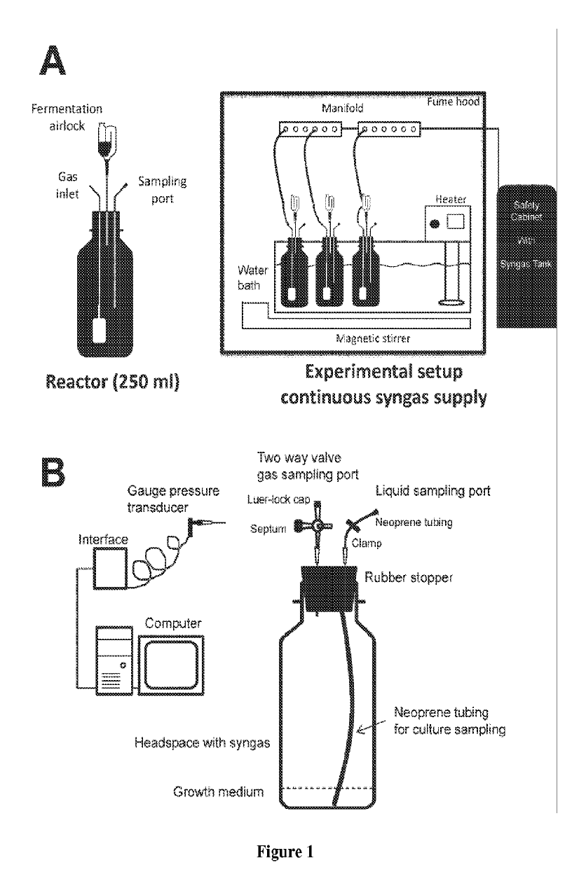

Experimental setup. The experiments to determine the ability of C. ljungdahlii to reduce propionic acid, n-butyric acid, n-valeric acid, n-caproic acid, or isobutyric acid (2-methyl propionic acid) to the corresponding alcohols were performed in triplicate in 250 ml rubber-stoppered Pyrex glass bottles (Fisher Scientific, Pittsburgh, Pa.), which were used as batch systems in regards to the growth medium, but with constant syngas supply (FIG. 1A). Three stainless steel needles of different length (21 gauge, Becton Dickinson Franklin Lakes, N.J., and ColeParmer, Vernon Hills, Ill.) were punched through each rubber stopper as gas supply, vent, and sample ports. The reactors were filled with 220 mL medium containing 0.5 g/L yeast extract and one of each carboxylic acids at a concentration of 15 mM, autoclaved, and then equipped with fermentation airlocks (www.winemakingsuperstore.com) filled with 5% sulfuric acid to prevent contamination by other microbes or oxygen, and placed in a temperature-controlled recirculating water bath for mechanical agitation using a 15 multi-position IKA magnetic stirrer (Cole-Parmer Vernon Hills, Ill. 60061 USA). Artificial syngas was constantly supplied to up to 12 reactors at a time, at a flow rate of 5 mL/min to each reactor with a gassing station with multiple outlets and low-flow brass needle valves (McMaster-Carr, Aurora, Ohio), sterile Masterflex viton tubing (Cole Parmer, Vernon Hills, Ill.), 0.22-μm sterile filters (Fisher Scientific), and sterile needles through the rubber stopper (size 6.5, VWR, Radnor, Pa.) connected to gas diffusion stones (Fisher Scientific). All reactors were inoculated with 2% exponentially growing pre-cultures. The fermentation was operated for ca. 400 h.

The stoichiometry of substrates and products of syngas fermentation with 0 mM n-butyric acid and 15 mM n-butyric acid was determined in 1 L rubber-stoppered Pyrex glass bottles filled with 68.4 ml medium without yeast extract and 1051.6 ml syngas headspace at a pressure of 28 psi. These batch studies, thus, had a finite amount of syngas (FIG. 1B). Here, yeast extract was omitted from the growth medium to minimize the interference of organic compounds (amino acids) in the fermentation. Two needles, each connected to a three-way valve, were used to take gas or liquid samples from the reactor. Gas pressure was measured using gauge pressure transducers (Model PX26, Omega Engineering, Inc., Stamford, Conn.) attached to hypodermic needles inserted through a rubber septum on the three-way valve of the sampling port. The transducers were connected to a data acquisition (DAQ) system interfaced with a personal computer with LabView® software (National Instruments Co., Austin, Tex.). Pressure data gathered by the DAQ system were converted to volume of gas at standard temperature and pressure (STP), according to the ideal gas law. Bottles were inoculated with 1% (vol/vol) exponentially growing preculture, and shaken at 100 RPM in an incubator at 35° C. Liquid and gas samples were taken daily. The fermentation was operated for ca. 200 h. Cell density (OD₆₀₀ₙₘ), pH, headspace gas pressure, concentrations of carbon monoxide, hydrogen, and carbon dioxide in the headspace, and concentrations of carboxylic acids and solvents in the culture medium were monitored daily. Each experimental condition was repeated twice (each time in triplicate). The experiment with the more consistent growth results was chosen for evaluation. One replicate of each experimental condition was not considered for calculations due to gas leakage, which became evident through pressure drops and decreased cell growth. Therefore, the results from 1 L bottle studies were obtained from duplicate experiments.

Analytical procedures. Cell dry weight concentration in the cultures was determined through measurement of the optical density with a Milton Roy Spectronic 1201 spectrophotometer at a wavelength of 600 nm after determining a correlation coefficient of 242 mg dry weight/(L*OD₆₀₀ₙₘ). The carbon content was calculated using the universal proportion of elements in microbial biomass (C₅H₇O₂N). A gas chromatography system (HP 5890, Hewlett Packard, Palo Alto, Calif.), which was equipped with a 7673 autoinjector and flame ionization detector, was used for the quantification of carboxylic acids and alcohols. GC columns were purchased from Sigma-Aldrich, Inc., St. Louis, Mo. The flow rates of hydrogen, air, and helium were 35, 380, and 30 mL/min. For the carboxylic acids, the column was a capillary GC column (Nukol); 15 m×0.53 mm i.d. (Supelco). The temperature program was 70° C. for 2 min, a ramp of 12° C./min to 200° C. where the temperature was held for 2 min. Injection port and detector were set at 200° C. and 275° C., respectively. For the quantification of alcohols, a custom-made packed bed glass column was used, 1.8 m×2 mm i.d. (Supelco). The support matrix of this column was Chromosorb W/AW80 over 100 mesh; phases were preconditioned: phase A was 10% Carbowax-20M; phase B was 0.1% phosphoric acid. Glass Purecol inlet liners, 2 mm i.d. were installed (Supelco). The inlet and detector temperatures were 220 and 240° C., respectively. The column temperature program was 100° C. for 2 min, a temperature ramp of 40° C./min to 180° C. where the temperature was kept for 5 min. Gas samples were analyzed with two Gow Mac gas chromatographs, series 580 (Bethlehem, Pa.), equipped with thermal conductivity detectors. For hydrogen quantification, a 4.5-m Supelco 60/80 Carboxen 1000 column at 25° C. and nitrogen as carrier gas were used. For carbon dioxide and carbon monoxide quantification, the gas chromatograph was equipped with a 1.8-m Supelco 80/100 Hayesep Q column at 25° C. and helium was the carrier gas.

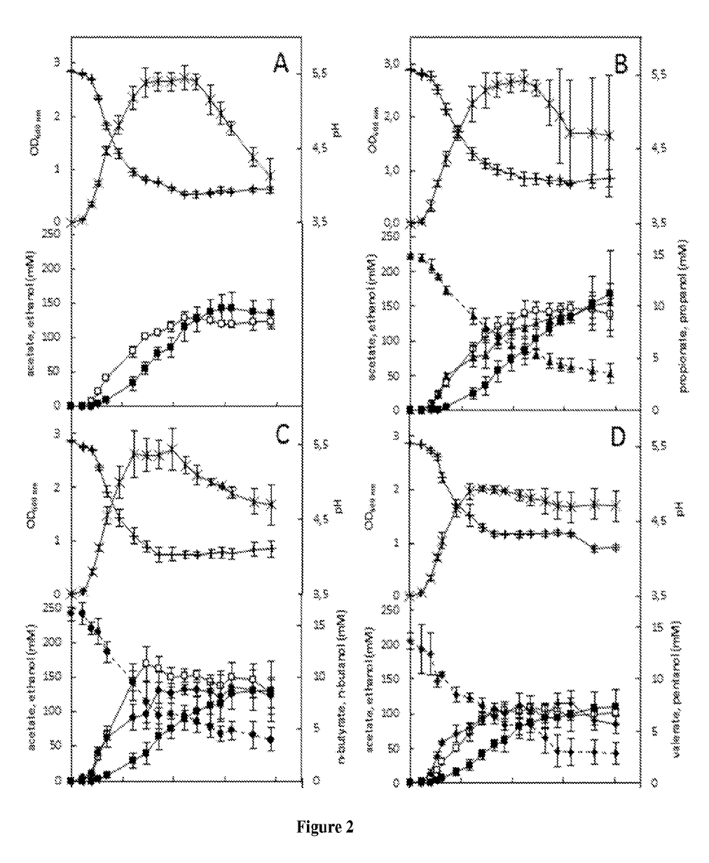

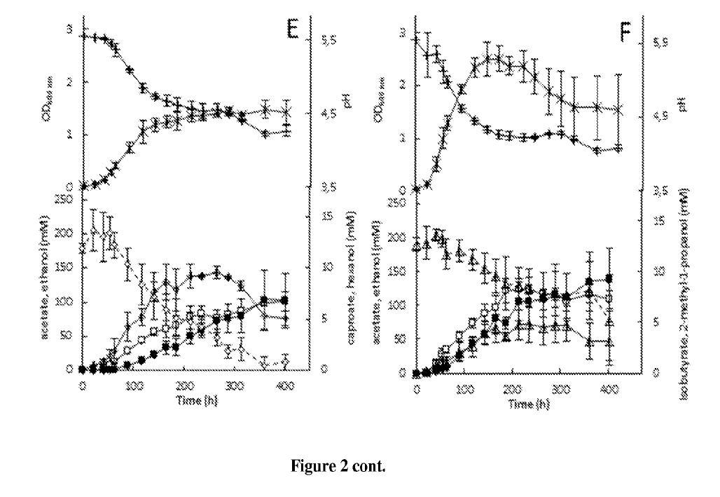

Results and Discussion. Conversion of carboxylic acids into alcohols. C. ljungdahlii ERI-2 was able to reduce short-chain carboxylic acids during continuous syngas supply by consumption of each carboxylic acid and production of their corresponding alcohol. Six conditions were tested: A) without carboxylic acid (negative control) (FIG. 2A); B) 15 mM propionic acid; C) 15 mM n-butyric acid (FIG. 2B); D) 15 mM n-valeric acid; E) 15 mM n-caproic acid; and F) 15 mM isobutyric acid. Growth was observed for all these 6 conditions (Table I). Cell yields and growth rates were similar for the negative control and the conditions with propionic acid and n-butyric acid. However, there was a tendency for the growth parameters to decrease with increasing carbon chain length of the added carboxylic acids (Table I), indicating an increased toxicity of the carboxylic acids with a longer carbon chain compared to the carboxylic acids with a shorter carbon chain. This is consistent with lower inhibitory concentrations of carboxylic acids with longer carbon chain lengths reported for C. thermocellum. Comparing isobutyric acid vs. n-butyric acid, it was found that the affinity of the reduction pathway to branched-chain carboxylic acids was lower than to linear-chain carboxylic acids, which is reflected in the lower amount of isobutyric acid consumed during the fermentation (Table I). The pH value in all experiments decreased from 5.5 to ca. 4.0 (Table I), with a final slight pH increase during the death phase of the cultures. The lowest final pH value was reached without carboxylic acid addition (Table I, FIG. 2A). Acetic acid and ethanol were produced during syngas fermentation with and without each of the tested carboxylic acids (Table I, FIG. 2A,B). The ratio of acetic acid vs. ethanol produced was close to 1 for all these six conditions.

An apparent efficiency was calculated for conversion of carboxylic acid into corresponding alcohol for each type; 92%, 68%, 52%, 46%, and 42% of the carbon amount of consumed propionic, n-butyric, n-valeric, n-caproic, and isobutyric acid were found in propanol, n-butanol, n-pentanol, n-hexanol, and isobutanol, respectively. It was hypothesized that the low carbon recovery values had been caused by gas stripping of the produced alcohols through the continuous flow of syngas, and not by inefficient cell metabolism. This was supported by the results of the stoichiometric experiments in which gas stripping was avoided in an n-butyric acid to n-butanol conversion with a finite amount of syngas in a pressurized 1 L bottle. Here, an equal amount of n-butyric acid was consumed compared to n-butanol produced during the experiment (0.31 mmol in Table II). Thus, the low conversion efficiencies in the experiments with constant syngas supply had been caused by gas stripping of the alcohol products. Gas stripping preferably removes alcohols over carboxylic acids, since alcohols are less polar, less soluble in water, more volatile, and have higher vapor pressures than the corresponding carboxylic acids.

Syngas experiments with C. ragsdalei P11 showed an ability to convert propionic acid and n-butyric acid into alcohols, albeit in lower concentrations and rates than C. ljungdahlii ERI-2. C. ragsdalei achieved a final concentration of 7.51 mM propanol and 4.81 mM n-butanol, while this was 10.4 mM propanol and 8.27 mM n-butanol for C. ljungdahlii ERI-2 (Table I). Both the concentrations of acetic acid and ethanol for C. ragsdalei were also considerably lower for each of three conditions studied, including the experiment without added carboxylic acid (Table I). The lower maximum cell density for C. ragsdalei compared to C. ljungdahlii is a likely reason for this inferior activity.

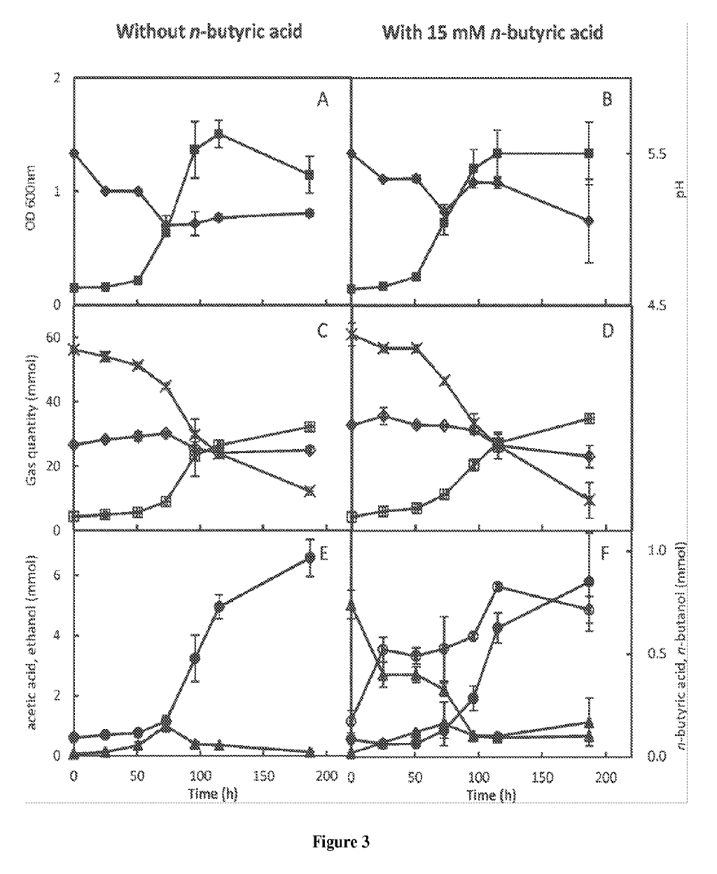

Fermentation and energy balances. Batch experiments with a finite amount of syngas in the headspace were conducted to obtain accurate stoichiometric equations (i.e., fermentation balances) for syngas fermentation without n-butyric acid or with 15 mM n-butyric acid. Growth curves were similar for both conditions until the end of the growth phase, which was at 115 h of the operating period (FIG. 3A,B). At the end of the growth phase, however, a lower pH of 5.1 was obtained without n-butyric acid compared to 5.3 with 15 mM n-butyric acid because of acidity loss due to n-butyric acid degradation (Table II; FIG. 3A,B). Gas quantity data showed that the culture consumed carbon monoxide and produced carbon dioxide throughout the operating period without much difference between conditions (FIG. 3C,D). n-Butanol production (0.6 mmol at 25 h) preceded ethanol production because of the initially higher n-butyric acid vs. acetic acid concentration, which made n-butyric acid a preferred electron acceptor at the start of the operating period (FIG. 3E,F). The maximum n-butanol concentration for the condition with n-butyric acid in the 1 L serum bottles was 13.3 mM, which was a total of 0.8 mmol at 115 h (FIG. 3F).

Substrate and product data for the period of growth, which occurred from 25 to 115 h after starting the experiment (FIG. 3A-F), were used to generate Table II. Next, data were normalized to one mol of ethanol to obtain the following empirical stoichiometric equations without (Eq. 1) and with n-butyric acid (Eq. 2):

7.1CO+1.0H₂→1.0 ethanol+0.1 acetic acid+5.0CO₂+0.4 cell carbon (1)

6.7CO+2.0H₂→1.0 ethanol+0.1 acetic acid+0.1 n-butyric acid+4.7CO₂+0.1 n-butanol+0.4 cell carbon (2)

A comparison between these fermentation balances shows that when normalized to ethanol, the relative hydrogen consumption for the conditions with n-butyric acid (2.0 mol) was double compared to without n-butyric acid (1.0 mol). In addition, carbon monoxide consumption and carbon dioxide production were somewhat lower with n-butyric acid compared to without, even though similar amounts of cell carbon (biomass) and acetic acid were formed (Eq. 1 and 2). It was hypothesized that these differences stem from the early availability of electron acceptor (n-butyric acid), which changes the ratio of redox mediators within the bacterial cell. Of note is that a relatively low percentage of ca. 2% of the electrons that were derived from the oxidation of carbon monoxide and hydrogen were used to reduce n-butyric acid to n-butanol. The two empirical equations are, therefore, somewhat similar. In addition, an increased availability of n-butyric acid would likely increase the ratio of n-butanol to ethanol, and thus optimize the process.

The two empirical stoichiometric equations are different from the theoretical stoichiometric equations that have been reported for acetate and ethanol formation because the latter equations are based on the presence of just carbon monoxide or carbon dioxide and hydrogen rather than a gas mixture that were used. This difference between the empirical and theoretical equations did not stem from a loss of carbon because the values of carbon recovery, which were calculated based on product formation vs. consumption of substrates, were 106% and 107% for both conditions, demonstrating that all substrates and products had been considered and that the reactors had no gas leak. In addition, no n-butyric acid was lost or metabolized by C. ljungdahlii in a pathway other than conversion to n-butanol because the conversion efficiency was on average 100.2%. However, not all electrons could be accounted for; the electron recovery in the products was 89% and 87% for without and with 15 mM n-butyric acid, respectively. One possible contributing loss could have been the diffusion of hydrogen gas through connectors and tubing.

To determine the amounts of energy that were contained, lost, and conserved in the substrates and products for evaluation of the energetic efficiency of the fermentation process, energy balances of the syngas fermentation without and with n-butyric acid to n-butanol conversion were calculated. Values for combustion energy contained in substrates and products were calculated from the standard Gibbs free energies of formation of all compounds after normalization to 1 mol of product (ethanol) formed by using Eq. 1 or Eq. 2 (Table II). Around 75% of the energy contained in the substrate gases and n-butyric acid was conserved in the fermentation products (i.e., acetate, ethanol, and n-butanol). 64% or 55% of substrate energy was conserved in ethanol (without or with n-butyric acid, respectively), and about 9% in n-butanol.

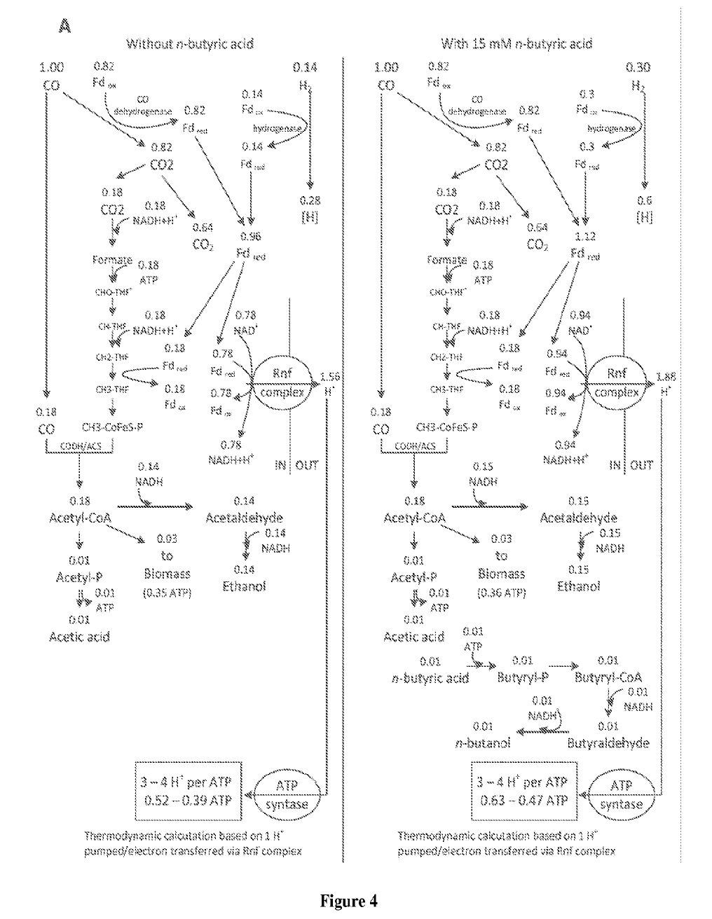

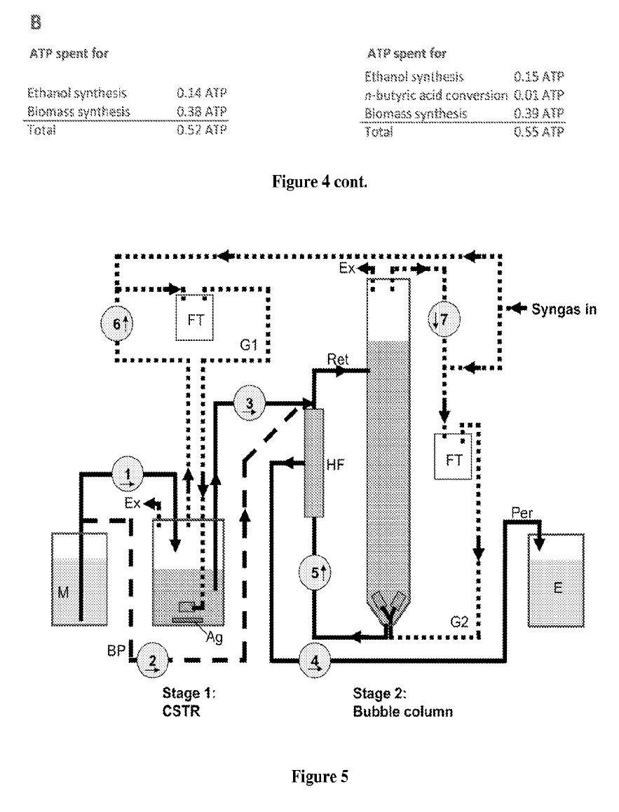

Metabolic model, ATP formation, and proton/electron ratio. The Wood Ljungdahl metabolic pathway that C. ljungdahlii uses to convert carbon monoxide into acetic acid or ethanol is well known. However, no data are available in the literature regarding how much ATP is produced per mol CO oxidized (ATP/CO yield), because the mechanism of ATP formation is still not completely understood. A metabolic model for carbon and electron flux was generated to calculate the ATP/CO yield, without and with 15 mM n-butyric acid present (FIG. 4A). Therefore, the ATP/CO yields have not been directly measured but rather are calculated here, using 2 independent methods. The results of both methods are compared and are useful to estimate how much metabolic energy C. ljungdahlii can generate from carbon monoxide fermentation. The metabolic pathway, the available knowledge about the mechanism of ATP formation, thermodynamic considerations, and the experimental data from hours 25-115 of the operating period (Table II) were integrated to build this metabolic model. For these calculations, data were normalized to 1 mol of carbon monoxide and hydrogen consumed from the syngas mixture (FIG. 4A,B).

During acetic acid production from carbon monoxide via the Wood-Ljungdahl metabolic pathway, no net ATP is spent or conserved via substrate level phosphorylation: one ATP is consumed in the methyl branch, and one ATP is conserved in the acetate kinase reaction during acetic acid formation from acetyl-CoA (FIG. 4A). For ethanol production from carbon monoxide, one net ATP is spent during substrate level phosphorylation per molecule of ethanol produced, because acetyl-CoA is reduced to ethanol and is not available as a source of ATP. Reduction of n-butyric acid to n-butanol requires one ATP for n-butyric acid activation via the carboxykinase/phosphotranscarboxylase pathway (with butyrate kinase and phosphotransbutyrylase enzymes) per mol of n-butyric acid reduced (FIG. 4A). For the metabolic model in FIG. 4A, it was assumed that alcohol formation always proceeds via the carboxykinase/phosphotranscarboxylase pathway, and that the AOR pathway is not present. It is discussed below what the energetic implications would have been when the AOR pathway is used. Regardless which of the two pathways is employed for alcohol production, it is always necessary for C. ljungdahlii to produce net ATP through a mechanism other than substrate level phosphorylation. The recently suggested mechanism involves the Rnf complex that generates a transmembrane proton gradient, using energy derived from electron transfer from reduced ferredoxin (Fd_{red}) to NAD⁺. The generated membrane potential is then used to phosphorylate ADP to ATP via a membrane-bound ATP-synthase. Synthesis of one mol ATP via ATP synthase requires a number of between 3 and 4 mol of protons translocated back into the cytoplasm, equaling a proton/ATP coefficient of 3 to 4. Therefore, to estimate how much ATP can be produced per CO oxidized, one must know how many electrons are available via Fd_{red} to the Rnf complex and what the proton/electron ratio is (i.e., how many protons does the Rnf complex pump across the cell membrane per electron transferred from Fd_{red} to NAD⁺).

The amounts of electrons available to the Rnf complex via Fd_{red} were calculated based on the knowledge that all electrons derived from oxidation of carbon monoxide and hydrogen are first transferred to ferredoxin via CO-dehydrogenase and hydrogenase, respectively (FIG. 4A), before they proceed to reduce metabolic intermediates with more positive redox potentials. Furthermore, a portion of the reduced ferredoxin (Fd_{red}) is required to reduce methylenetetrahydrofolate to methyltetrahydrofolate in the methyl branch of the Wood-Ljungdahl pathway (FIG. 4A). The remaining part of the Fd_{red} is then available to the Rnf complex for generation of a transmembrane proton motive force and consecutive ATP synthesis via a membrane-bound ATP synthase.

No experimental results, but only theoretical calculations, are reported in the literature about the stoichiometry of protons pumped vs. electrons transferred from Fd_{red} to NAD⁺ via the Rnf complex. The redox couples Fd_{red}/Fdₒₓ (−420 mV) and NADH₂/NAD (−320 mV) have a redox potential difference of 100 mV, which is equivalent to an energy difference of −20 kJ/mol. Müller et al. speculated that this energy difference would be enough to pump only one cation across the cell membrane in Acetobacterium woodii, resulting in a proton/electron ratio of 0.5. For their calculation, they assumed an electrochemical ion potential of −200 mV for A. woodii, but did not state from which source this value was obtained. For C. ljungdahlii, no actual data are available, but it is reasonable to assume that its electrochemical ion potential is close to that of Clostridium acetobutylicum, which is a related bacterium for which actual data are available for the transmembrane pH gradient and the electrical potential for different pH values in the growth medium. Using their data, the energy AG required for translocation of 1 mol of protons was calculated for external pH values of 6.5 and 4.5 (Eq. 3).

ΔG=2.303*RT*log(c2/c1)+ZFΔV (3)

where R is the ideal gas constant (8.315*10⁻³ kJ*mol⁻¹*K⁻¹), T is temperature (308 K), log(c2/c1) is ΔpH (0.2 for external pH of 6.5, and 1.5 for external pH of 4.5), Z is charge of the protons (+1), F is Faraday constant (96.49 kJ*mol⁻¹*V⁻¹), and ΔV is electric membrane potential in Volt of the destination side of the proton (−0.09 V and 0.00 V for an external pH of 6.5 and 4.6, respectively). AG values calculated were 9.9 kJ/mol per proton for an external pH of 6.5 and 8.90 kJ/mol for external pH of 4.5. These values suggest that the −20 kJ energy derived via transfer of 2 electrons from Fd_{red} to NAD⁺ is sufficient to pump 2 protons across the membrane. Therefore, it was concluded that the proton/electron ratio can be up to 1.0. Considering a proton/ATP coefficient of 3-4, this means that oxidation of 2 mol Fd_{red} can theoretically produce 1.0-1.3 mol of ATP.

Based on this consideration and the above energetic calculation, the model suggests that a total of 0.39 to 0.52 mol of ATP was produced per mol of CO consumed without n-butyric acid, and between 0.47 to 0.63 mol ATP per mol of CO with 15 mM n-butyric acid (FIG. 4A). Overall, assuming a proton/electron ratio of 1.0, the results suggest an ATP/CO yield of ca. 0.5. If one assumed a proton/electron ratio of only 0.5, which was suggested by Müller et al (2008), then the ATP/CO yields calculated with the model would only be in the range of ca. 0.25 ATP/CO. To evaluate which assumption is more realistic (1 vs. 0.5 protons per electron), theoretical values of ATP spent (i.e., needed) for ethanol and biomass synthesis were used (for without n-butyric acid) and for ethanol and biomass synthesis plus n-butyric conversion (for with n-butyric acid) (FIG. 4B). Bacteria have to spend energy for synthesizing biomass from carbon and other macronutrient elements. The energy necessary to synthesize biomass is inversely proportional to the carbon content of the carbon source. For synthesis of 10 g dry weight (DW) of biomass, approximately one mol of ATP is necessary when the carbon source contains 6 carbon atoms per molecule, which is the case with glucose. Consequently, cells growing on substrates, such as CO or CO₂, need approximately 6 mol of ATP to synthesize 10 g DW of biomass. It was calculated from the fermentation data (Table II) that the cells must have spent 0.52 ATP/CO for synthesis of ethanol and biomass without n-butyric acid, and 0.55 ATP/CO for synthesis of ethanol, n-butanol, and biomass with 15 mM n-butyric acid (FIG. 4B). These values are well in agreement with the results derived from the metabolic model, assuming a proton/electron ratio of 1.0. Both calculations suggest an ATP/CO yield of ca. 0.5 (FIG. 4A,B). Note that data were normalized to one mole of CO although hydrogen was consumed, and that, therefore, the ATP/CO yield would be ca. 20% lower in experiments when only carbon monoxide is present (without hydrogen consumption).

In the case that the earlier-mentioned AOR pathway is responsible for conversion of carboxylic acids into their corresponding alcohols rather than the carboxykinase/phosphotranscarboxylase pathway, then 1 mol ATP less would be consumed per mol of alcohol produced. But also, per mol of alcohol produced, one mol of Fd_{red} less would be available to the Rnf complex. In that case, the ranges for ATP produced per CO consumed, which were calculated using the metabolic model (similar to FIG. 4A), would be 0.32-0.43 and 0.39-0.52 without and with n-butyric acid, respectively. With the method to calculate ATP spent (similar to FIG. 4B), the ATP/CO yield would be 0.38 and 0.39, respectively. Regardless, which of the two pathways are used for carboxylic acid to alcohol conversion, the ATP/CO yield is ca. 0.4-0.5. The assumption of a proton/electron ratio of 1.0 always provided the best explanation for the fermentation results. If one assumed a stoichiometry of only 0.5 protons/electron, as previously suggested, the ATP production calculated using the model would be only at 50% of above ranges, and would not sufficiently explain the observed yields of biomass and alcohols.

It is anticipated that n-butyrate to n-butanol conversion via syngas fermentation can be improved considerably by preventing the formation of the undesired by-products acetate and ethanol. To improve the energy conservation in n-butanol at the expense of ethanol is to optimize the n-butyric acid feeding method to divert electron flow to butyryl-CoA instead to acetyl-CoA. At the same time, n-butanol should be constantly removed to avoid end product inhibition. Another strategy is to use only hydrogen gas and to eliminate carbon monoxide. The results indicate that the number of protons pumped per electron transferred from ferredoxin to NAD⁺ via the Rnf complex is 1.0. Therefore, hydrogen via hydrogenase, Fd_{red}, Rnf complex, and ATP synthase, can theoretically provide the required stoichiometry of 1 ATP and 2 NADH₂ to activate and reduce n-butyric acid to n-butanol. By replacing carbon monoxide with hydrogen for conversion of n-butyric acid into n-butanol, formation of by-products such as acetic acid, ethanol, and carbon dioxide would likely be eliminated. The final strategy is to knock out the methyl-branch of the Wood-Ljungdahl pathway (or at least a part of it). This would prevent electrons from reducing CO₂ to the methyl group, and prevent production of the precursor acetyl-CoA. Instead, the electrons are forced to reduce externally provided n-butyric acid into n-butanol. Such a genetically modified strain would only be viable when supplied with external n-butyric acid as an electron acceptor, because it would otherwise not be able to maintain its redox balance. The viability of such a strain would also depend on the amount of ATP equivalents spent for the reduction of n-butyrate to n-butanol (i.e., on the AOR-pathway being used for n-butyric acid conversion).

Here, it is shown that the carboxydotrophic bacteria C. ljungdahlii ERI-2 and C. ragsdalei P11 can produce n-butanol or other alcohols during syngas fermentation when external n-butyric acid or their corresponding carboxylic acids are provided. 13.3 mM of n-butanol (91% of theoretical yield from 14.6 mM initial concentration of n-butyric acid) was produced with nonoptimized n-butyric acid addition, suggesting that optimization would improve this conversion considerably. The enzymatic machinery for the conversion of the carboxyl group into an alcohol group possesses a broad specificity for carboxylic acids of different carbon chain length and branching characteristics, resulting in the production of n-propanol, n-butanol, n-pentanol, n-hexanol, and isobutanol. Carboxydotrophic bacteria are a favorable biocatalyst for the reduction of short-chain carboxylic acids into alcohols due to their high substrate and product specificity and their promise to be genetically modified to repress side product formation from syngas fermentation.

TABLE I
Parameters for syngas fermentation by C. ljungdahlii ERI-2 and C. ragsdalei P11 in medium amended without and with 15 mM carboxylic
acids of different carbon chain length. Values were obtained from triplicate batch cultures with a constant supply of syngas.

| | no. of | | | | | produced at end of experiment (mM) | | |
|---|---|---|---|---|---|---|---|---|
| | carbon atoms | max. opt. | growth | | consumed | alcohol from | final | |
| | in carboxylic | density at | rate | final | carboxylic | carboxylic | acetic | final |
| condition | acid | 600 nm | (h⁻¹) | pH | acid (mM) | acid | acid | ethanol |

C. ljungdahlii ERI-2

| | | | | | | | | |
|---|---|---|---|---|---|---|---|---|
| no carboxylic | NA | 2.73 ± 0.24 | 0.094 ± 0.001 | 3.95 ± 0.04 | NA | NA | 123.27 ± 10.52 | 135.53 ± 20.10 |
| acid | | | | | | | | |
| propionic acid | 3 | 2.69 ± 0.19 | 0.084 ± 0.012 | 4.11 ± 0.11 | 11.26 ± 1.03 | 10.44 ± 1.69 | 138.02 ± 22.99 | 169.05 ± 61.65 |
| | | | | | | n-propanol | | |
| n-butyric acid | 4 | 2.71 ± 0.39 | 0.094 ± 0.004 | 4.10 ± 0.11 | 12.13 ± 1.79 | 8.27 ± 1.75 | 126.36 ± 20.85 | 129.87 ± 43.23 |
| | | | | | | n-butanol | | |
| n-valeric acid | 5 | 2.01 ± 0.04 | 0.077 ± 0.007 | 4.16 ± 0.03 | 10.88 ± 1.07 | 5.63 ± 0.92 | 100.48 ± 5.61 | 109.58 ± 25.88 |
| | | | | | | n-pentanol | | |
| n-caproic acid | 6 | 1.47 ± 0.17 | 0.049 ± 0.003 | 4.26 ± 0.07 | 11.11 ± 1.18 | 5.11 ± 0.68 | 101.10 ± 14.31 | 102.58 ± 39.98 |
| | | | | | | n-hexanol | | |
| isobutyric acid | 4 | 2.50 ± 0.31 | 0.077 ± 0.008 | 4.08 ± 0.04 | 7.47 ± 4.03 | 3.14 ± 1.89 | 112.90 ± 12.99 | 137.76 ± 46.10 |
| | | | | | | isobutanol | | |

C. ragsdalei P11

| | | | | | | | | |
|---|---|---|---|---|---|---|---|---|
| no carboxylic | NA | 0.68 ± 0.10 | 0.052 ± 0.001 | 4.99 ± 0.07 | NA | NA | 31.30 ± 3.15 | 59.65 ± 0.66 |
| acid | | | | | | | | |
| propionic acid | 3 | 0.53 ± 0.14 | 0.093 ± 0.024 | 5.23 ± 0.15 | 10.39 ± 2.05 | 7.51 ± 2.01 | 39.43 ± 8.39 | 48.08 ± 24.17 |
| n-butyric acid | 4 | 0.98 ± 0.05 | 0.120 ± 0.004 | 4.83 ± 0.10 | 6.85 ± 1.23 | 4.81 ± 1.55 | 64.13 ± 11.64 | 24.87 ± 9.75 |

TABLE II
Fermentation data of batch syngas fermentations without and with
15 mM n-butyric acid added for the time period from 25 to 115 h.

| | without | with 15 mM |
|---|---|---|
| | n-butyric acid | n-butyric acid |
| maximum OD₆₀₀ | 1.51 ± 0.12 | 1.34 ± 0.21 |
| cell mass DW (mg) | 18.93 ± 1.85 | 16.65 ± 2.99 |
| carbon fixed in cell mass DW (mmol) | 1.58 ± 0.15 | 1.39 ± 0.25 |
| pH after 115 h | 5.08 ± 0.02 | 5.31 ± 0.04 |
| ATP for biomass synthesis (mmol) | 11.36 ± 1.11 | 9.99 ± 1.79 |
| carbon monoxide consumed (mmol) | 30.11 ± 2.09 | 25.90 ± 3.13 |
| hydrogen consumed (mmol) | 4.19 ± 0.89 | 7.81 ± 3.90 |
| n-butyric acid consumed (mmol) | NA | 0.31 ± 0.05 |
| n-butyric acid consumed (%) | NA | 76.3 ± 0.30 |
| carbon dioxide produced (mmol) | 21.51 ± 2.16 | 18.19 ± 2.29 |
| acetic acid produced (mmol) | 0.23 ± 0.01 | 0.22 ± 0.07 |
| ethanol produced (mmol) | 4.25 ± 0.42 | 3.84 ± 0.52 |
| n-butanol produced (mmol) | NA | 0.31 ± 0.04 |
| ATP for alcohol production (mmol) | 4.25 ± 0.42 | 4.15 ± 0.57 |
| overall carbon recovery (%) | 106.3 ± 3.1 | 106.7 ± 1.4 |
| combustion energy (kJ) in all substrates | 2054 | 2387 |
| combustion energy (kJ) in all products | 1545 | 1756 |
| combustion energy (kJ) in ethanol | 1324 | 1324 |
| combustion energy (kJ) in n-butanol | NA | 207 |

Abbreviations: OD₆₀₀ = optical density at 600 nm; DW = dry weight; values given in mmol are the total amounts of substrates and products consumed or produced; or ATP consumed. Percent carbon recovery considers all substrates, products and carbon fixed in biomass. Energy data (kJ) are normalized to 1 mol of ethanol produced and based on Eq. 1 or Eq. 2.

Syngas, which is a blend of carbon monoxide, hydrogen and carbon dioxide, was studied as an economical source of energy and electrons with pure cultures of Clostridium ljungdahlii as a biocatalyst for the carboxylic acids reduction. Acetic acid, propionic acid, n-butyric acid, isobutyric acid, n-valeric acid, and n-caproic acid were successfully converted into their corresponding alcohols. Furthermore, biomass yields and fermentation stoichiometry from the experimental data enabled us to amend thermodynamic calculations with the goal to evaluate how much metabolic energy C. ljungdahlii can generate during fermentation of carbon monoxide. The results show ATP yield of 0.42 ATP per carbon monoxide molecule consumed for fermentation of syngas, and 0.38 ATP per carbon monoxide molecule consumed when n-butyrate is added to the growth medium. The ratio of protons pumped across the cell membrane vs. electrons transferred from ferredoxin to NAD⁺ via the RNF complex is suggested to be 1.0. The results obtained in this example suggest that the production of alcohols, based on the reduction of carboxylic acids, may be an attractive alternative industrial process.

In this example, the ability of Clostridium ljungdahlii to convert organic acids into the corresponding alcohols using syngas as a source of energy and reducing power was tested. Also, stoichiometry of the conversion was investigated and carbon, electrons and energy flux was studied. The general conclusions are presented.

Clostridium ljungdahlii is Able to Convert Organic Acids of Different Molecular Weight into the Corresponding Alcohols Using Syngas as Source of Energy and Reducing Power.

Results obtained from cultures of Clostridium ljungdahlii tested in presence of propionic acid, n-butyric acid, n-valeric acid, n-caproaic acid, and isobutyric acid using syngas as a source of energy and reducing power showed that the bacterium has the enzymatic machinery to reduce carboxylic acids to the corresponding alcohols. Even though it lacks of some enzymes necessary for carbon chain elongation, it can produce alcohols with a carbon chain length longer than two carbons when the corresponding carboxylic acid is provided. Alcohols produced in presence of carboxylic acids contain the same number of carbons of the carboxylic acid supplied in the growth medium, showing that there is no transformation of the carbon chain, but only the acid group.

The enzymatic machinery has a broad specificity for carboxylic group, being able to reduce carboxylic acids of different carbon chain length. Addition of carboxylic acids to the growth medium stimulates H₂ consumption and production of alcohols. This stimulation has application in the industry to increase the utilization of the H₂ present in the syngas and to obtain more specific fermentation products. CO could be replaced by H₂ in non-growing cultures to convert carboxylic acids into the corresponding alcohols preventing the production of other byproducts.

Rnf Complex in Clostridium ljungdahlii is Able to Pump 2H⁺ Out of the Membrane Per Fd_{red}:NAD⁺ Oxidoreduction Reaction.

The energy balance supports the hypothesis of two protons are pumped out of the cell membrane by Rnf complex per ferredoxin:NAD⁺ oxidoreduction reaction. No data has been published before related to the function of the Rnf complex.

Experimental setup for continuous syngas fermentation experiments. The system was designed to be able to run reproducible experiments with several different conditions at the same time. It consists of a water bath placed on a multi position magnetic stirrer in order to provide consistent temperature control and agitation. The syngas was conducted from a gas tank placed into a safety cabinet using ¼ in stainless steel tubbing, which was connected to two manifolds in a row with six outlets each manifold, totalizing twelve outlets. Each outlet consists of a hose barbed connector, connected to a ⅛ in neoprene tubbing with a luer-lock connector at the end.

Reactors were made of 250-ml capacity media bottles. A rubber stopper was used to close the top opening. The stopper was held with an open cap and a washer. A needle through the stopper was used as the syngas inlet and inside the bottle was connected to a neoprene tube with a sparging stone attached at the end. A second needle was used as the exhaust and was connected to a gas trap in order to prevent oxygen to come in. Finally, a 6 in needle was used to take liquid samples. This needle was closed with a luer-lock cap when not sampling (FIG. 1A).

Experimental setup for batch syngas experiments. This experimental setup was designed to run syngas fermentation experiments in batch cultures with limited amount of syngas. The reactors are gastight and allow headspace pressure measurement as well as gas sampling for gas composition analysis. Liquid samples can also be taken for cell density, pH, and product concentration measurement. The reactors consist of a 1120 ml bottle with a rubber stopper in the top opening secured by a metal washer and a screw cap ring in order to keep it pressurized. One needle punched through the rubber stopper was used to take liquid samples. The needle was connected to a neoprene tubing in order to reach the bottom of the reactor for sampling. The outer part of the needle was connected to a 2 in long neoprene tubing and closed with a luer-lock cap at the end. The neoprene tubing was secured with a clamp to control the liquid flow when taking the samples because of the over pressure in the inside of the reactor (FIG. 1B).

A second needle that was punched through the rubber stopper was used to measure inside gas pressure and take gas samples for measuring its composition. The outer part of the needle was connected to a 2-way valve. One inlet of the valve was used to take the gas samples and measure the gas pressure and was equipped with a rubber septum. The other inlet was closed with a luer-lock cap (FIG. 1B). Gage pressure transducers (Model PX26, Omega Engineering, Inc.) which were attached to hypodermic needles, were used to measure the pressure inside the reactor. The pressure transducers were connected to a computer through an interface, and the pressure data converted to volume gas at standard temperature and pressure (STP), according to the ideal law of gases.

The reactors were placed on a shaker into an incubator and the temperature was set at 35° C. At the moment of the gas pressure sampling, the shaker was stopped and the gage pressure transducers connected to the two way valve by punching the needle through the septum. The shaker was stopped to prevent gas leakage through the septum when the needle was connected. To equilibrate the temperature after opening the incubator's door, half an hour after the connection of the pressure transducers the pressure was recorded. 500 micro liter of gas was sampled using a gastight Hamilton sample lock glass syringe and analyzed using gas chromatography. The pressure in the syringe was equilibrated with the ambient pressure by placing the extreme of the needle in a beaker with water and unlocking the syringe, then locking it again to keep the sample inside. The sample was injected in the GC immediately to avoid leakage.

Culture of Clostridium ljungdahlii in serum bottles for inoculum source purpose. Medium preparation (ATCC 1756) Stock solutions for minerals, vitamins, and trace metals were prepared separately.

Mineral stock solution. For 1 liter solution, pour 900 ml DI water in a beaker and add the following chemicals:

sodium chloride 80 g

ammonium chloride 100 g

potassium chloride 10 g

potassium phosphate monobasic 10 g

magnesium sulfate 20 g

calcium chloride 4 g

Adjust the final volume to 1 L with DI water

Vitamin stock solution. For 1 liter solution, pour 900 ml DI water in a beaker and add the following chemicals:

pyridoxine 0.01 g

thiamine 0.005 g

riboflavin 0.005 g

calcium pantothenate 0.005 g

thioctic acid 0.005 g

amino benzoic acid 0.005 g

nicotinic acid 0.005 g

vitamin B12 0.005 g

biotin 0.002 g

folic acid 0.002 g

MESNA 0.01 g

Adjust the volume to 1 L with DI water

Trace metals stock solution. For 1 liter solution, pour 900 ml DI water in a beaker and add the following chemicals:

nitrilo triacetic acid 2 g

manganese sulfate 1 g

ferrous ammonium sulfate 0.8 g

cobalt chloride 0.2 g

zinc sulfate 0.2 g

copper chloride 0.02 g

nickel chloride 0.02 g

sodium molybdate 0.02 g

sodium selenite 0.02 g

sodium tungstate 0.02 g

Adjust the volume to 1 L with DI water

Medium preparation. For a 250-ml total capacity serum bottle (20 ml of medium and 230 ml headspace), pour 15 ml DI water in a beaker and add the following solutions and chemicals:

Mineral solution 0.6 ml (30 ml/L)

Trace metals solution 0.2 ml (10 ml/L)

Yeast extract 0.01 g (0.5 g/L)

MES sodium salt 0.65 g (32.58 g/L or 150 mM)

Adjust pH to 5.5 by adding KOH 5 M solution
Add DI water to complete 19.6 ml
Pour the solution in the serum bottle (250 ml) and flush the medium with nitrogen for 20 min using a long (6 in) needle and a rubber stopper in the opening to prevent air to come in. Take the needle out and seal the bottle with a crimp seal. Connect the bottle to a syngas outlet using a needle and flush the headspace for 5 min using another needle as an exhaust. After 5 min take the exhaust needle out and let the pressure equilibrate with the pressure in the line (14 psi). Autoclave the bottle (Set the time according to the autoclave instructions)
Let the bottle and medium cool down and add the following solutions (always work close to a flame or in a biological hood. Clean the stopper with ethanol before inserting a needle):

Vitamin solution 0.2 ml (10 ml/L)

Cysteine sulfide 2.5% 2.2 ml (10 ml/L)

Let the cysteine sulfide work for 5 min before inoculation.
Inoculate 10% v/v with a living culture.

Medium preparation for precultures without yeast extract. Experiments where no yeast extract is used require a preculture grown without yeast extract in growth medium to prevent any remaining yeast extract to contaminate the experiment. For this purpose, prepare the growth medium following the same instructions explained above, except the addition of yeast extract in the solution.

### Example 2

This example shows a two-stage continuous fermentation process for production of ethanol from synthesis gas (syngas) with Clostridium ljungdahlii. The system consists of a 1-L continuously stirred tank reactor as a growth stage and a 4-L bubble column equipped with a cell recycle module as an ethanol production stage. Operating conditions in both stages were optimized for the respective purpose (growth in stage one and alcohol formation in stage two). The system was fed with an artificial syngas mixture, mimicking the composition of syngas derived from lignocellulosic biomass (60% CO, 35% H₂, and 5% CO₂). Gas recycling was used to increase the contact area and retention time of gas in the liquid phase, improving mass transfer and metabolic rates. In stage two, the biocatalyst was maintained at high cell densities of up to 10 g DW/L. Ethanol was continuously produced at concentrations of up to 450 mM (2.1%) and ethanol production rates of up to 0.37 g/(L*h). Foam control was essential to maintain reactor stability. A stoichiometric evaluation of the optimized process revealed that the recovery of carbon and hydrogen from the carbon monoxide and hydrogen provided in the ethanol produced were 28% and 74%, respectively.

TABLE III
Performance parameters of continuous operation of the 2-stage system
at time point 1517 h at which stable operating conditions had been
achieved. These parameters were used to calculate the fermentation
balance explained in the text. The gas inlets for stage 1 and 2 contained
the same gas, and are therefore summarized in one column.

| | CONCENTRATIONS | | |
|---|---|---|---|
| | Outlet stage | Outlet stage | Inlet stage |
| Compound | 1 | 2 | 1 & 2 |
| CO (G), (vol %) | 53 | 19 | 60 |
| H₂ (G), (vol %) | 34 | 14 | 35 |
| CO₂ (G), (vol %) | 13 | 63 | 5 |
| Ethanol (L), (mM) | 11.5 | 428.4 | NA |
| Acetic acid (L), (mM) | 146.5 | 142.5 | NA |
| Bacteria (gDW/L) | 0.476 | 9.34 | NA |

| | RATES (mmol/(L*min) | | |
|---|---|---|---|
| Compounds | Stage 1 | Stage 2 | Total |
| CO in | 0.607 | 0.808 | 0.768 |
| CO out | 0.330 | 0.110 | 0.154 |
| CO consumption | 0.277 | 0.698 | 0.614 |
| H₂ in | 0.354 | 0.471 | 0.448 |
| H₂ out | 0.182 | 0.085 | 0.105 |
| H₂ consumption | 0.172 | 0.386 | 0.343 |
| CO₂ in | 0.051 | 0.067 | 0.064 |
| CO₂ out | 0.085 | 0.371 | 0.314 |
| CO₂ production | 0.034 | 0.303 | 0.250 |
| Ethanol production | 0.007 | 0.136 | 0.110 |
| Acetic acid production | 0.094 | 0.025 | 0.039 |

| | EFFICIENCIES (%) | | |
|---|---|---|---|
| | Stage 1 | Stage 2 | Total |
| CO consumption | 46 | 86 | 80 |
| H₂ consumption | 49 | 82 | 77 |

Abbreviations: (G) and (L) indicate gas or liquid state at ambient conditions; gDW/L: gram dry weight per liter.

Biocatalyst and growth conditions. C. ljungdahlii ERI-2 (ATCC 55380) was used as a biocatalyst, since it had proven to be a good ethanol producer. Bacteria were always grown anaerobically at 35° C. in medium designed for efficient syngas fermentation, which is referred to here as 1× medium. Precultures were grown in 160-mL serum bottles containing 10 mL of 1× medium adjusted to pH 5.5, and syngas in the headspace at a pressure of 1.93 bar. Precultures were maintained by weekly transfer of 2% (vol/vol). The concentration of MES (2-(N-morpholino)ethanesulfonic acid) buffer was 5 g/L in the precultures, and in the initial startup medium in the 1-L CSTR fermentor, where yeast extract was added at 0.05 g/L to promote initial growth. Yeast extract and MES were omitted from medium in stage two, and from the continuous feed medium in which the pH was controlled via addition of 2 M KOH or HCl. Prior to inoculation of stage one, stage one and two were filled with 1 L and 4 L of 1× concentrated growth medium, respectively. The pH setpoints in stage one were 5.5 (low) and 5.7 (high), with the actual medium pH always being at the low end of the range due to acidogenesis. In stage two, the pH setpoints were 4.4 (low) to prevent acid crash in case the culture turned acidogenic, and 4.8 (high) to prevent the culture from turning acidogenic in the first place. In the sourcemedium for continuous operation, the concentration of all minerals, trace elements, and vitamins was doubled (2× medium) or quadrupled (4× medium), after a maximum OD₆₀₀ had been reached in stage two with 1× medium. Antifoam 204 (Sigma-Aldrich) was added to the medium reservoir at 10 μL/L, which prevented foaming in stage one. In stage two, because of high cell densities, a foam controller (Cole Parmer, Vernon Hills, Ill.) was installed to deliver antifoam 204 solution (100× diluted) on demand. The antifoam amounts and concentrations had been carefully determined in previous experiments to provide efficient foam control without killing the cells by adding too much of the agent, which seems to be toxic to C. ljungdahlii. In stage two, a total of 462.5 mL of 100× diluted Antifoam 204 was consumed during the entire run at an average rate of 0.236 mL/h.

Reactor Setup.

The two-stage continuous system was set up according to FIG. 5. The stage one fermentor was a 2-L Braun Biostat M CSTR (Braun, Allentown, Pa.) with 1 L working volume. The agitation speed was 200 rpm. Stage two was a custom-made 6-L bubble column with 4 L working volume. Both systems were equipped with temperature (water jacket), and pH control. Stage two was equipped with a foam control system (Cole Parmer) that injected 100× diluted antifoam 204 solution upon detection of high foam levels. Peristaltic media pumps (Cole Parmer, Vernon Hills, Ill.) #1-4 and gas-recycle pump #7 were operated at variable flow-rate, while the cell recycle pump #5 and gas recycle pump #6 were set to 180 mL/min. Microbubble spargers (MoreFlavor, Concord, Calif.) were made of stainless steel with a pore size of 0.5 μm. Foam traps in the gas recycle lines prevented clogging of microspargers. The rates of syngas supply into both stages were maintained at levels that exceeded the consumption by at least 10% to avoid limitation of gaseous substrate, which has been reported to be detrimental for ethanol production. Flexible tubing (Cole Parmer) was Norprene for liquid lines, and Viton for the gas lines, respectively, since Viton has the lowest numbers for gas permeability. The cell recycle module was a Cellflo polyethersulfone hollow fiber module with 500-cm² membrane surface area and 0.2-μm pore size (C22E-011-01N, Spectrum Laboratories, Inc., Rancho Dominguez, Calif.).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1. A method for obtaining a product comprising a substituted or unsubstituted C₃ to C₁₀ alcohol comprising the steps of:
a) providing in a vessel a mixture comprising i) a substituted or unsubstituted C₃ to C₁₀ carboxylic acid, ii) Clostridia species, and a gaseous composition comprising: carbon monoxide, carbon monoxide and hydrogen, or carbon dioxide and hydrogen; and
b) maintaining the reaction conditions under anaerobic conditions such that the substituted or unsubstituted C₃ to C₁₀ alcohol is formed in the mixture, and at least 40% of the carbon from the exogenous substituted or unsubstituted C₃ to C₁₀ carboxylic acid is recovered as alcohol formed by the Clostridia species,
wherein the steps a), and b) are carried out in the absence of viologen dyes, anthraquinone and other quinone dyes, triphenylmethane dyes, phthalocyanines, methane dyes, pyrrole dyes, pteridines and pteridones, flavines, and metal complexes of metals of secondary groups VI, VII, and VIII.

2. The method of claim 1, further comprising step c) separating at least a portion of the substituted or unsubstituted C₃ to C₁₀ alcohol from the mixture.

3. The method of claim 1, wherein the Clostridia species is one in which the Wood-Ljungdahl pathway is non-functional.

4. The method of claim 3, wherein the Clostridia species lacks a functional enzyme selected from the group consisting of formate dehydrogenase, formyl-THF synthetase, methyl-THF cyclohydrogenase, methylene-THF reductase, methyltransferase, Acetyl-CoA synthase, and combinations thereof.

5. The method of claim 1, wherein the Clostridia species is Clostridium ljungdahlii ERI-2, Clostridium ljungdahlii PETC, Clostridium ljungdahlii C-01, Clostridium ragsdalei P11, Clostridium autoethanogenum, Clostridium carboxidivorans, Clostridium coskatii, or a combination thereof.

6. The method claim 1, wherein step b) is conducted at 20° C. to 45° C. and a pH of from 4.0 to 6.5.

7. The method claim 6, wherein step b) is conducted at 30° C. to 40° C. and a pH of from 4.0 to 6.5.

8. The method claim 6, wherein step b) is conducted at 35° C. to 37° C. and a pH of from 4.0 to 6.5.

9. The method of claim 1, wherein step b) is carried out in two stages, wherein the pH for the first stage is maintained at from 5.0 to 6.5 and the pH for the second stage is maintained at from 4.0 to 5.5.

10. The method of claim 1, wherein at least 40%, 50%, 60%, 70%, 80%, or 90% of the carbon in the carboxylic acid is recovered in the alcohol.

11. The method of claim 1, wherein the substituted or unsubstituted C₃ to C₁₀ carboxylic acid is propionic acid, n-butyric acid, n-valeric acid, n-caproic acid, enanthic acid, caprylic acid, pelargonic acid, capric acid, isobutyric acid, benzoic acid, phenylacetic acid, phenylpropionic acid, phenylbutyric acid, 3-methoxy-4-hydroxybenzoic acid, 3-methoxy-4-hydroxyphenylacetic acid, cinnamic acid, lactic acid, 2-methylbutyrate, 2-methyl-2-buteneoate, glutaric acid, succinic acid, adipic acid or a combination thereof.

12. The method of claim 1, wherein the substituted or unsubstituted C₃ to C₁₀ alcohol is n-propanol, n-butanol, n-pentanol, n-hexanol, n-heptanol, n-octanol, n-nonanol, n-decanol, isobutanol, benzyl alcohol, 4-phenylbutan-1-ol, (E)-3-phenylprop-2-en-1-ol, 4-(hydroxymethyl)-2-methoxyphenol, 2-phenylethanol, 4-(2-hydroxyethyl)-2-methoxyphenol, 3-phenylpropan-1-ol, propane-1,2-diol, (E)-2-methylbut-2-en-1-ol, 2-methylbutan-1-ol, pentane-1,5-diol, butane-1,4-diol, hexane-1,6-diol, or a combination thereof.

13. The method of claim 1, wherein the method is carried out continuously or semi-continuously.

14. The method of claim 1, wherein step b) is carried out for at least 24 hours.

15. The method of claim 14, wherein step b) is carried out for from 1 to 90 days.
